(19) United States
(12) Patent Application Publication
(10) Pub. No.: US 2021/0137180 A1
(43) Pub. Date: May 13, 2021

(54) Personal Mobile Air Heater

(71) Applicants: John Jerome Kusnierek, Springfield, OH (US); Jeffrey Richard DeWispelaere, Elk River, MN (US); Frank Edward Kaczmarek, Ivanhoe, MN (US)

(72) Inventors: John Jerome Kusnierek, Springfield, OH (US); Jeffrey Richard DeWispelaere, Elk River, MN (US); Frank Edward Kaczmarek, Ivanhoe, MN (US)

(21) Appl. No.: 16/679,230

(22) Filed: Nov. 10, 2019

(51) Int. Cl. A41D 13/005; H05B 1/02; A41D 13/002; A41D 1/02; A41D 1/06; A41D 19/00; A62B 17/00; A61M 16/10; A61G 11/00; A41D 27/28; A43B 7/02; D06F 58/26

(52) U.S. Cl. CPC A41D 13/0051; H05B 1/0297; A41D 13/0025; A41D 13/0053; A41D 1/02; A41D 1/06; H05B 2203/02; A62B 17/005; A61M 16/1075; A61G 11/00; A41D 27/28; A43B 7/02; D06F 58/26; A41D 19/00

(57) ABSTRACT

A self-contained functional and physical architecture is described to utilize a honeycomb configuration Positive Temperature Coefficient semiconductor thermistor heater to provide breathable warm air to an individual under cold weather conditions. The high-temperature of the heater element provides substantial efficiency allowing hours long use on battery power only. The invention utilizes pulsed heater operation in combination with lengthwise air mixing to rapidly and efficiently lower the temperature of the high temperature heated air to a temperature that is breathable. The invention solves issues with visor fogging, adaptability to a wide temperature range, and adaptability to a wide range of individual exertion. This system is applicable to a wide range of cold weather work environments, cold weather sports, scientific exploration, military operations, or medical needs. Several physical embodiments are described utilizing various forms of helmets and masks.

### BACKGROUND

### Field of the Invention

Cold Weather Personal Equipment, Medical Equipment, Sports Equipment, Breathing Apparatus

### Prior Art

### US Patents

| Pat. No. | Kind Code | Issue Date | Patentee |
|---|---|---|---|
| 2,784,714 | | 1957 Mar. 12 | Pitzipio |
| 3,249,108 | | 1966 May 12 | Terman |
| 3,491,754 | | 1970 Jan. 27 | Weese |
| 4,245,146 | | 1981 Jan. 13 | Shioi |
| 4,412,537 | | 1983 Nov. 1 | Tiger |
| 4,492,228 | | 1985 Jan. 8 | Makovic |
| 4,610,247 | | 1986 Sep. 9 | Stroup |
| 4,620,537 | | 1986 Nov. 4 | Brown |
| 4,793,343 | | 1988 Dec. 27 | Cummins |
| 4,245,631 | | 1981 Jan. 20 | Wilkenson |

The present invention relates generally to personal equipment that can provide warm breathable air in cold ambient conditions and more specifically to a self-contained system comprising an integrated system of pulsed air heating, continuous air flow, closed loop control, and battery electrical power.

There are a number of circumstances where it is desirable for medical, comfort, task performance, or emergency reasons to have breathable warmed air within cold ambient air environments while the wearer of such a system is freely mobile. For example, construction workers, oil field workers, farm workers, or military operations can benefit while performing tasks in cold weather by having the present invention provide breathable warmed air in a self-contained system that allows unrestricted mobility. In the case of outdoor sports such as skiing and snowmobiling, having warm air to breath can provide increased comfort or the ability to enjoy sports over a broader range of conditions for longer periods of time. In the medical field, existing literature shows various medical risks associated with breathing cold air which include angina, asthmatic attacks, coronary events, and pneumonia depending on age, health, and other factors.

The present invention provides a complete and self-contained solution to the problem of providing breathable warm air in cold weather conditions while a person is highly mobile and performing various tasks free of any external power or air supply systems. The present invention herein applies to multiple fields such as, but not limited to, construction personal equipment, recreational sports equipment, military equipment, and medical respiratory devices.

The prior art addressing this need can be broken into two main approaches. First of these approaches is a face mask with a heater element located within the mask area where warm air is provided by directing ambient cold airflow across the heating element. The required airflow over the heater element is provided by the respiratory inhalation and exhalation action of the wearer of the apparatus as shown in U.S. Pat. No. 2,784,714 Pitzipio, U.S. Pat. No. 3,249,108 Terman, U.S. Pat. No. 3,491,754 Weese, U.S. Pat. No. 4,412,537 Tiger, U.S. Pat. No. 4,492,228 Makovic, U.S. Pat. No. 4,610,247 Stroup, U.S. Pat. No. 4,620,537 Brown, U.S. Pat. No. 4,793,343 Cummins, or U.S. Pat. No. 4,245,631 Wilkenson. The heater element in prior art may be electrical such as a power transistor bonded to a metal plate or chemical such as catalytic or combustion.

A second approach is a breathing apparatus that provides warmed air by means guiding cold inhaled air through passages that are placed closely to some areas of the wearers body which are already warm such as the head or chest of the wearer's anatomy as shown in U.S. Pat. No. 3,491,754 Weese, U.S. Pat. No. 4,412,537 Tiger, U.S. Pat. No. 4,492,228 Makovic, or U.S. Pat. No. 4,610,247 Stroup. The passages thus placed conduct body heat into the cold air flowing through the passages to warm the air flow before inhalation. Similar to the first approach, the wearers own breathing action provides the airflow across the heating section of the apparatus. In both approaches there may be provisions such as valving to manage the moisture contained in exhaled breath by directing it outside of the apparatus.

In prior art approaches, achieving a practical level of performance across a range of ambient conditions is greatly hampered by lack of heating efficiency, lack of continuous airflow, and limited control means. Thus, prior art embodiments have one or more of the following disadvantages: lack of performance across a variety of weather conditions, lack of control of the temperature of the warmed inhaled air, poor temperature stability of the warmed inhaled air, resistance to the wearers breathing action while both inhaling and exhaling, lack of efficiency of the heater element both in power and air flow loss, short mission times with batteries due to lack of heater efficiency, an undesirable cooling effect on the users body if cold air passages are run next to the body, and an accumulation of condensation, water droplets, or frost inside or outside of the apparatus. The present invention solves all of these problems and has the ability to provide warm breathable air in a self-contained wearable and portable system while providing a means to control the inhaled air temperature across a wide range of weather conditions for several hours while simultaneously controlling moisture accumulation. It is applicable to a variety of configurations corresponding to a wide range of intended uses. This includes physical embodiments described herein such as a fully enclosed impact resistant helmet, enclosed bubble helmet for increased visibility, closed mask for increased mobility, and open mask for high exertion situations. From several prototype tests, the present invention fully meets design objectives and will provide warm breathable air in a self-contained wearable and portable system while providing a means to control the inhaled temperature across a wide range of weather conditions for several hours while controlling moisture accumulation.

The present invention can also be used to provide warm air to bath the face, head, hands, or feet as well as providing warm breathing air.

The present invention can also be used to provide warm air to the inside of a user's jacket, shirt, gloves, trousers, or boots.

The present invention can also be used to provide warm air to small enclosures such as incubators, pet beds, or shoe racks.

### SUMMARY OF THE INVENTION

It is an object of this invention to provide an efficient physical and functional architecture for heating of cold ambient air by means of continuous airflow directed across a Positive Temperature Coefficient (PTC) semiconductor thermistor heater whereby the heated air is subsequently cooled to breathable temperatures in an individually wearable system with the heater in close proximity to the users nose and mouth.

It is an object of this invention to contain the said physical and functional architecture in a self-contained wearable mobile system which provides several hours of use free of any external power supply, air supply, or control system.

It is an object of this invention to provide an efficient physical and functional architecture for the heating of cold ambient air in which moisture accumulation inside of the personal mobile air heater is eliminated as an issue.

It is an object of this invention to provide a control algorithm for the heating of cold air by use of a positive temperature coefficient semiconductor thermistor heater using pulsing to heat cold air that is subsequently cooled to breathable temperatures all contained in a wearable mobile system which actively directs air through the heater then to the user and is actively controlled with a temperature feedback system.

It is an object of this invention to provide an electrical architecture which utilizes the physical architecture, functional architecture, and control algorithms for the heating of cold air by use of a positive temperature coefficient semiconductor thermistor heater using pulsing to heat cold air that is subsequently cooled to breathable temperatures all contained in a wearable mobile system which actively directs air through the heater then to the user and is actively controlled with a temperature feedback system.

It is an object of this invention to provide a physical embodiment of the said architecture, control system, and battery which is added onto a high impact helmet.

It is an object of this invention to provide a physical embodiment of the said architecture, control system, and battery which is integrated into a high impact helmet.

It is an object of this invention to provide a physical embodiment of the said architecture, control system, and battery which is integrated into a high visibility bubble helmet.

It is an object of this invention to provide a physical embodiment of the said architecture, control system, and battery which is used with a closed mask.

It is an object of this invention to provide a physical embodiment of the said architecture, control system, and battery which is used with an open mask.

### Advantages

Accordingly, several advantages of one or more aspects of the new embodiment are as follows:
a system that operates at high temperatures using a PTC semiconductor thermistor heater that provides substantial system efficiency in order to provide multiple hours of operating time using high density rechargeable batteries,
a means to use a high temperature PTC semiconductor thermistor heater in close physical proximity to the users mouth and nose to provide breathable warm air in cold weather conditions,
a system that is self-contained, wearable, and free of any external power or air supplies,
a physical and functional architecture for a personal mobile air heater that provides the wearer controlled warm breathable air across a variety of weather conditions in a single system,
a physical and functional architecture for a personal mobile air heater that virtually eliminates moisture accumulation in or outside of the mobile personal air heater to include the breathing apparatus and helmet visors,
a physical and functional architecture for a personal mobile air heater that may be applied as a modular accessory to existing masks, helmets, and other headgear,
a physical and functional architecture for a personal mobile air heater that may be applied as integral part of a mask, helmet, or other headgear,
a physical and functional architecture which can utilize several types of displays for the user,
a flexible physical and functional architecture which can also be used to provide warm dry air to the inside of a user's jacket, shirt, gloves, trousers, or boots, and,
a flexible physical and functional architecture which can also be used to provide warm dry air to small enclosures such as incubators, nests, or shoe racks.

### Conclusion, Ramifications, and Scope

Accordingly, the reader will see that the personal mobile air heater invention described herein is a substantive breakthrough in the field of personal equipment. It is enabled by the use of a high temperature heater which is pulsed to gain sufficient efficiency to provide useful operation times in a self-contained unit. In turn, the high temperature efficiency of this system inherently enables control of the air temperature provided to the user over a wide range of external ambient air temperatures. Importantly, the invention also inherently provides dry air to the user to eliminate moisture condensation inside the invention which eliminates a major problem in prior art.

The present invention can be utilized wherever a person experiences cold ambient air temperature in order to improve comfort, safety, performance, or mitigate medical conditions. Furthermore, the personal mobile air heater has additional advantages of being easily manufacturable, adaptable to a variety of price points, adaptable to a variety of material technologies, and can be adapted to a wide range of embodiments such as the entire system contained in a helmet or the entire system distributed over the user's body

Although the description above contains many specificities, these should not be construed as limiting the scope of the embodiments but as merely providing illustrations of some of several embodiments. For an example, the physical and functional architecture may be adapted to heat the inside of jackets, gloves, pants, and boots with warm dry air. For additional example, the physical and functional architecture may be adapted to provide warm dry air for medical or incubator needs. The scope of the embodiments should be determined by the appended claims and their legal equivalents, rather than by the examples given.

### DRAWINGS—REFERENCE NUMERALS

FIG. 1 is a plot of the resistance verses heater surface temperature characteristic of a Positive Thermal Coefficient (PTC) thermistor semiconductor heater element.

FIG. 2 is an orthogonal view of a PTC heater element in a honeycomb arrangement with a plurality of channels.

FIG. 3 is a side view in section of the personal mobile air heater subsystem based on the honeycomb PTC heater.

FIG. 4 is the functional architecture of the personal mobile air heater system.

FIG. 5 is the electrical architecture of the personal mobile air heater system.

FIG. 6 is the control law of the personal mobile air heater system.

FIG. 7 is an illustration of the power verses time trace for the PTC heater pulses for extreme cold temperature of the personal mobile air heater system.

FIG. 8 is an illustration of the power verses time trace for the PTC heater pulses for moderate temperature of the personal mobile air heater system.

FIG. 9 is a plot of the air temperature verses time at various locations in the personal mobile air heater subsystem.

FIG. 10 is a top view of a personal mobile air heater in the physical configuration of an add-on to a high impact helmet showing key features.

FIG. 11 is a left-hand view of a personal mobile air heater in the physical configuration of an add-on to a high impact helmet showing key features.

FIG. 12 is a right-hand view of a personal mobile air heater in the physical configuration of an add-on to a high impact helmet showing key features.

FIG. 13 is a front view of the entire personal mobile air heater as worn by a user.

FIG. 14 is a top view of a personal mobile air heater in the physical configuration of an integrated high impact helmet showing key features.

FIG. 15 is a left-hand view of a personal mobile air heater in the physical configuration of wide field-of-view helmet.

FIG. 16 is a front view of a personal mobile air heater in the physical configuration of a closed mask.

FIG. 17 is a front view showing details of the closed mask.

FIG. 18 is a front view of a personal mobile air heater in the physical configuration of an open mask.

FIG. 19 is a front view showing details of the open mask.

FIG. 20 is view of a typical control panel for the personal mobile air heater.

FIG. 21 is a view of a typical add-on display for the personal mobile air heater.

FIG. 22 is a view of a typical integral display for the personal mobile air heater.

### DETAILED DESCRIPTION

This embodiment applies Positive Temperature Coefficient (PTC) semiconductor thermistor heaters in an individually wearable and self-contained system that provides breathable warm air to the wearer. This embodiment includes the application of one or more PTC heaters in a heater subsystem, one or more energy dense batteries in a power subsystem, a compact positive air flow subsystem, and a control subsystem specific to this embodiment. Continuous air flow is integral to the use of PTC heaters for this application. The continuous air flow combined with PTC temperature also dries the air and so virtually eliminates moisture accumulation as an issue in or outside of physical embodiment.

All or part of the embodiment is adaptable to various application specific physical configurations for the purpose of providing controlled temperature warm air to an individual user. For an ambient temperature range below approximately 60 F, this embodiment has no fundamental limitation to the design intent of providing warm dry air to an individual user in a system that is self-contained and provides unrestricted mobility. The application specific design objective temperature ranges can be narrower, broader, lower, or higher. For example, an application specific embodiment intended for artic outdoor petroleum work might be −90 F to 0 F, while a different application for outdoor farm work might be +20 F to +55 F.

FIG. 1 shows that for a PTC semiconductor thermistor heater, electrical resistance is low up until a critical surface temperature T_{c} (1) and then the resistance increases non-linearly up to resistance (2). At the PTC critical surface temperature T_{c}, the change in resistance can be as high as 50% for each degree Fahrenheit surface temperature and so temperature T_{c} is sometimes called the switching temperature or the trip temperature. Using Ohm's law of I=V/R, where I is current, V is voltage, and R is resistance, this means that further increases in current past the critical temperature are effectively stopped and hence the electrical energy being turned into heat cannot be further increased and is stable. Therefore, for a constant airflow, PTC heaters are self-regulating in that the heat energy is limited to that produced with the electrical conditions at the critical temperature T_{c}. The PTC resistance-to-temperature relationship is unlike nichrome-based heaters which have a more linear relationship between resistance and temperature.

For the purpose of illustrating efficiency of the embodiment herein, an ambient temperature range of minus 50 Fahrenheit (F) to plus 50 F is arbitrarily chosen. In an electric air heater, efficiency is determined by the temperature differential between the inlet air temperature and the heater element, the heat exchanger surface area, the air flow losses inside the heater passages, the air density, and the air flow velocity. PTC heaters used in this invention can provide a temperature T_{c} of between 300 F to 400 F surface temperature for the heater element due to the physics of their materials. Therefore, this provides a substantial temperature differential of 250 F to 450 F between the ambient air temperatures illustrated above and the PTC heater element. By contrast, a typical nichrome-based heater used to provide warm air would have a far lower temperature differential in this application of 50 F to 150 F in order to provide a breathable temperature air. The larger temperature differential of the PTC heater provides improved heater efficiency over heater elements operating at a lower temperature differential. In the present invention, the PTC heater is several times more efficient than a nichrome resistive heater system.

However, the air thus heated by a PTC heater is far too hot to breathe. The embodiment contained in this invention solves this issue and enables the use of PTC heaters to efficiently create breathable warm air which enables mobile personal air heater system weights and volumes such that the invention can be placed into a self-contained and lightweight wearable system worn by an individual without the need for any external power or support systems.

Fundamental to this embodiment is that the time constant for a PTC heater is much shorter than that for a nichrome heater. For the parameters of the personal mobile air heater with a 600 Watt PTC heater, the useable time constant is on the order of 0.015 seconds to 0.030 seconds. This short time constant is caused by the very low resistance of the PTC heater element below T_{c} and the resulting current in rush until the heater element reaches temperature T_{c}. The short time constant of the PTC heater element enables the personal mobile air heater to use the PTC heater effectively as a pulsed air heater. A similar set of parameters for a standard nichrome resistive heater would have a time constant that is 50 to 100 times longer than a PTC heater. Thus, the PTC heater can take advantage of the efficiency of operating at higher temperature by using pulse modulation to accurately control air temperature to a lower average output temperature.

In the Personal Mobile Air Heater, the PTC heater configuration is a symmetrical honeycomb of heater surface arranged streamwise to the airflow directed through it as shown in FIG. 2. Ambient cold air (7) enters the PTC heater (3) and flows over a multiplicity of honeycomb heater channels (6) and exits as heated air at (8). The electrical current flow goes into the conductive area at (4) and returns through conductive area at (5). This physical heater configuration further increases efficiency of the heater subsystem with a large surface area to volume ratio while at the same time providing low air flow losses. This honeycomb heater element configuration would be difficult to economically create with nichrome resistive heater technology.

The combination of high surface area of the PTC heater element and high surface temperature also dries the air to low humidity levels. Thus, the honeycomb PTC heater element enables the invention herein to provide dry air to the user. This dry air easily absorbs the humidity from the users exhaled breath and thereby eliminates fogging or moisture accumulation on the inside of the system. This is a major advantage of the present invention over prior art.

Finally, PTC heaters sized for this embodiment are energy dense and will typically have a maximum of 600 Watts of output heat energy with a weight of 0.5 lb for a power density of over 1200 Watts per pound. They are also energy volume dense with a 600 Watt system available in energy volume densities of over 60 Watts per cubic inch.

In this invention, the PTC heater element is used in a PTC based air heater subsystem as shown in FIG. 3 to provide breathable warm air to the user. Cold ambient air flows through a cold duct such as a flexible hose (9) into a reducer fitting (12) which serves the purposes of attaching the cold air channel to the air warmer, isolating the cold air channel thermally, providing a thermocouple mounting, and as required by a specific design, adapting the air channel cross-sectional shape to the PTC heater cross-section area. While FIG. 3 shows a round cross-sectional shape, the embodiment is applicable to a wide variety of cross-sectional shapes. Threaded boss (11) provides a means for a threaded fastener (10) to engage with the cold ambient air channel and retain it. This embodiment is not limited to the one retention means shown as there are a variety of air channel retention mechanisms possible which include adhesives, twist-lock, and a threaded air channel. The attributes that can be considered include manufacturing cost, durability, temperature, and whether the PTC element should be exchangeable. The reducer fitting can be made with electrically conductive or non-conductive materials, but needs to be capable of operation at up to 400 F without loss of structural strength or out-gassing of the material. The ambient temperature Tₐ (22) is measured by an industry standard thermocouple (54) the output of which is used in the control subsystem.

The reducer fitting (12) then connects with a cold side PTC conductor section (25) which is electrically conductive. The cold side PTC conductor section (25) physically retains the reducer fitting (12) with threaded boss (24) into which threaded fastener (23) is used to engage the reducer fitting (12) and retain it. Electrical power is brought in through wire (13) and attached to threaded boss (15) conductively contiguous with (25). The electrical power wire is retained by threaded fastener (14). The PTC heater element (5) is retained inside of (25) by electrically conductive epoxy (26). The PTC heater element is then electrically connected to a hot side conductor section (29) which also uses conductive epoxy (26) and includes an electric power wire (17) attached at threaded boss (16) and retained by threaded fastener (18).

The PTC heater electrical path comprises electrical power brought in through wire (13) flowing through cold side conductor section (25), flowing though circumferentially arranged conductive epoxy (26), flowing through the PTC heater element (5), through the second circumferentially arranged conductive epoxy (26), through the hot side conductor section (29), and finally through wire (17). This embodiment is not limited to this means of electrically mounting a PTC heater as there are a variety of means to electrically mount the PTC heater element which include circumferential pressure clips, longitudinal pressure clips, welding, brazing, soldering, or threaded fastener. The attributes that can be considered in a specific design for electrically mount the PTC heater include manufacturing cost, durability, temperature, and whether the PTC element should be exchangeable.

The air temperature immediately downstream of the PTC heater at temperature T₀₁ (30) is the hottest air temperature in the present invention and too hot to breathe at temperatures of over 300 F. The hot side conductive section (29) includes an integral aerodynamic mixer (68) to provide turbulent mixing of hot and cold air pulses by promoting turbulence. The action of the air mixer (68) reduces the air temperature to T₀₂ (32), but it is still too hot to safely inhale. Additional duct length Lₑ (31) is required to turbulently mix the hot and cold air pulses to reduce the average air temperature to T_{f} which is safely breathable. Thus, the mixer (68) works with the pulsed heater to mix alternating cold and hot air pulses to reduce the average temperature in the shortest possible hot side duct length Lₑ (31). For efficiency, the duct length from the PTC heater to the person breathing the warm air, should be as short as possible to reduce heat losses to cold external air. The duct length Lₑ (31) can be as short as four inches in practice. In the case of the PTC heater used in this embodiment, this means bringing a maximum air temperature down by as much as 300 F to an average air temperature ranging between 65 F to 75 F.

The resulting warmed air flows through into a mixer duct (21) which serves the purposes of attaching the hot side electrical conductor (29) to an adapter for cross-sectional shape, isolating the hot air channel thermally, and mounting a thermocouple sensor. While FIG. 3 shows a round cross-sectional shape, the embodiment is applicable to a wide variety of cross-sectional shapes. By varying its length and shape, the warm air channel (21) also opens up options for where to place the PTC heater element for system weight balance on the wearer or to move the PTC to a more convenient locations on the wearer. Threaded boss (20) provides a means for a threaded fastener (19) to engage with the hot air channel and retain it. This embodiment is not limited to the one retention means shown as there are a variety of air channel retention mechanisms possible which include adhesives, twist-lock, and a threaded air channel. The attributes that can be considered include manufacturing cost, durability, temperature, and whether the PTC element should be exchangeable. The warm air channel can be made with electrically conductive or non-conductive materials, but needs to be capable of operation at up to 400 F without out-gassing of the material. The warmed air temperature Tf (33) is measured by an industry standard thermocouple (58) mounted in threaded boss (35) with sensor wire (36). The output of thermocouple (58) is used in the control system.

The PTC Heater element (5), cold side PTC conductor section (25), hot side conductive section (29) can be expected to reach surface temperatures as high as 400 F and pose both a potential thermal safety hazard and a loss in efficiency by heat loss to the atmosphere. Therefore insulation (56) is arranged circumferentially around PTC heater to prevent thermal injuries and reduce heat losses to the atmosphere. In addition to thermal properties, this insulation can be selected based on cost, volume, ease of manufacturing, durability, water resistance, and other design specific considerations.

The PTC breathable air warmer subsystem is integrated into a mobile personal air heater system functional architecture as shown in FIG. 4. Cold ambient air external to the system at point (37) is at a state of velocity zero and illustrative temperature ranging between minus 50 F and plus 50 F. This cold ambient air is drawn into the system through air filter (51) and reaches velocity and temperature state (38) by the action of fan (52). The air filter can consistent of any combination of debris screen, precipitation filter, dust filter, or chemical filter depending on the application. The specific filtering used is a sizing consideration for fan (52).

The fan driven airflow in this system is a constant airflow system for user comfort and elimination of moisture accumulation inside of the system. Fan (52) is functionally connected to the controller (49) and may be of any type such as axial, centrifugal out-flow, or centrifugal in-flow depending on specific design needs. Upon exiting the fan, air velocity and temperature state (39) is achieved which is approximately 25 feet per second velocity for a typical closed helmet system. The air velocity is increased to velocity state (55) by flowing through reducer section (53) which serves the purpose of increasing the air velocity and reducing the cross-sectional area to that required for the cold air channel (9). For a typical system design with an axial fan, the area ratio of the reducer section is approximately six to one. The cold air channel may be of constant or variable cross-sectional area depending on the specific design. The cold air in velocity state (22) then flows by cold side temperature sensor (54) which is also functionally connected to the controller (49) and is measuring ambient temperature Tₐ. The air then flows into the reducer section (12) leading to some loss in air velocity in state (27).

The air then flows through the PTC based heater comprising the PTC heater element (5), cold side PTC conductor section (25), and hot side conductive section (29). The air velocity is relatively constant through the PTC heater, but the air temperature rises rapidly to at least 300 F at state (30) depending on whether the PTC heater is pulsed on or off. Due to the heater pulsing, the air temperature state (30) is time dependent. After air state (30), the mixer (68) turbulently blends hot air pulses with cold air pulses to reduce the average air temperature to state (33) at the end of hot air duct (21). The air temperature of the hot side is measured by temperature sensor (58) which is also functionally connected to the controller (49) and is measuring temperature final T_{f}.

As shown, there is an air velocity loss across the mixer as shown from state (30) to state (33) which must be accounted for in the system design. An air channel (59) which may take the form of an expander, vent, or mask then directs the warmed air to the vicinity of the wearer's mouth and nose with air state (67). There is a slight reduction in air velocity and temperature due to turning losses, turbulence losses, and conductive losses. A typical target air temperature at air state (67) is 70 F for breathing by the wearer, but this is application dependent and also dependent on user settings on the controller.

For mobility, the system draws all power from one or more energy dense rechargeable batteries (50) to allow free mobility for several hours. For specific designs, the battery may be rechargeable, replaceable, or supplemented by an electrical cable tying into vehicle power, fixed outlets, or generators. The power from battery (50) is functionally connected to the controller (49) which controls power to the fan (52), PTC heater (5), displays (69), and the controller (49) itself. The wearer provides inputs to the system through control panel (48).

One embodiment of an associated electrical architecture of the present invention is shown in FIG. 5. A high-density rechargeable battery (50) provides all system electrical power and is electrically connected to the controller (49) via wire pair (65). Connector (92) provides a means to quickly change batteries and electrically connect the battery to a charger. Control knob (73) provides a means for the user to turn the device on and set the temperature. Other functionality may be added such as a fan only function for cold air delivered to the user, a test function for system self-test, and a selection of display mode.

The controller subsystem (49) contains all system control algorithms, analogue-to-digital conversions, and power supplies to make the system fully controllable. A temperature sensor used by the control subsystem is the cold side temperature sensor (54) with wire pair (61) providing the electrical signal between the controller and temperature sensor. A second temperature sensor used by the control subsystem is the hot side temperature sensor (58) with wire pair (63) providing the electrical signal between the controller and temperature sensor. Additional sensors for air temperature, PTC temperature, air mass flow, oxygen fraction, fan speed, and voltage may be added in application specific designs.

The controller provides electrical power to the PTC heater cold side electrical connection (15) and hot side electrical connector (16) via wire pair (62). The system has been demonstrated to operate with one PTC heater, but more than one PTC heater may be used depending on the application specific design needs. The controller provides electrical power to fan (52) shown here as an axial fan via wire pair (66). Finally, the controller provides signals to display (69) via signal wire (64).

Connectors may be added to this system at various locations to aid with system manufacturability, in-field maintainability, and user convenience while donning the system.

This electrical architecture also provides substantial flexibility to the arrangement and placement of the sub-systems in application specific designs. This includes application specific designs which are entirely self-contained inside of a helmet or designs in with the subsystems distributed over the wearers body.

The control algorithm shown in FIG. 6 provides two modes of pulsing the PTC air heater subsystem depending on the average heat energy required to bring the average air temperature to a user controlled warmed air temperature. Due to the electrical resistance relationship to PTC heater surface temperature, a voltage control approach cannot be used as such a system is unstable below Tc. The present invention takes into account the minimum time pulse and the associated heat energy in the minimum PTC heater pulse because this drives the need to change control modes. The control algorithm described here is for an average adult over a wide range of ambient temperature conditions. Smaller PTC heaters can reduce the minimum heat energy in a pulse, but the PTC heater size required for an average adult will lead to a physically smaller heater which can create excessive air flow losses.

In the control algorithm, first the control knob (73) gain G is read. Next the temperature final T_{f} (33) is read from the hot side temperature sensor (58). Next the temperature ambient Tₐ (22) is read from the cold side temperature sensor (54). Next temperature ambient Tₐ (22) is compared to a threshold temperature Tₘ which is the temperature at which the control mode switches between variable pulse time-on and fixed time-off for lower temperature ambient conditions requiring more heater energy or fixed pulse time-on and variable time-off for moderate temperature ambient conditions requiring less heater energy.

The first control mode is that if temperature ambient Tₐ is lower than threshold temperature Tₘ, then to achieve comfortable breathable air, the variable pulse time-on tₐ is calculated as a proportional gain G times the temperature differential between Tₐ and T_{f}. The resulting tᵤ pulse is then provided to the PTC heater at the end of which there is a preset fixed time-off t_{z}. The algorithm then repeats at rate of 1 to 5 Hertz.

For the second control mode where temperature ambient Tₐ is greater than threshold temperature Tₘ, less heat energy is required and a fixed time-on pulse tₘ is provided to the PTC heater. The fixed time-on pulse tₘ is the minimum pulse width achievable with the PTC heater element to reach the critical temperature T_{c} and thereby maintain heater efficiency. The variable pulse time-off tₒ is calculated by applying proportional gain G to the temperature differential between Tₐ and T_{f}. After the fixed time-on pulse tₘ has ended, the PTC is then held in an off condition for tₒ time and then the algorithm repeats at end of time period tₒ.

The two control modes shown allow the PTC heater subsystem to be pulsed to achieve a comfortable breathable air temperature across a wide range of temperatures. For example, a personal mobile air heater system designed to operate between conditions of −50 F to +50 F would require that both modes be available. For an application specific design over a narrower temperature range, a single control mode can be used for simplicity.

FIG. 7 shows the pulses for the first control mode where tᵤ (43) is a variable time-on pulse being modulated by the gain G applied to the temperature differential between Tₐ and T_{f} while the time-off t_{z} (44) is fixed. FIG. 8 shows the pulses of the second control mode where tₒ (46) is a variable time-off pulse being modulated by the gain Gₘ applied to the temperature differential between Tₐ and T_{f} while the time-on tₘ (45) is fixed. Depending on the application specific needs, a control mode can be designed where both time-on and time-off are variable.

FIG. 9 illustrates the effect of the heater energy pulses on the air temperature flowing through the system verses time. In the temperature verses time trace shown, the time-on and time-off pulses are equal and fixed to illustrate the pulsed heater in the invention. The ambient temperature air (22) is shown as a constant and temperature line (41) is the maximum safe breathable air temperature. The maximum air temperature T₀₁ attained is at air state (30) immediately downstream of the PTC heater and is far higher than that which is safe to breath for a human user. The heater pulsing allows flow path lengthwise air mixing in which a cold air pulse is followed by a hot air pulse and by turbulence a mixing action occurs between the hot and cold pulses reducing the average air temperature. Immediately after the mixer (68) which can take the form of fixed radial stators or a simple screen, the average air temperature Toe (32) can be seen to be reduced, but still oscillating with the peak temperature being out of phase with the peak temperature (30) proportional to the air velocity. However, the air temperature Toe at (32) is still too hot to breathe. Mixer duct (21) provides the necessary lengthwise air flow space for the turbulence to further reduce the average air temperature to temperature T_{f}(33) which is safe to breath. The control system will see this temperature as T_{f}(33) from the hot side temperature sensor (58). The total hot side duct length Lₑ (31) is an important design consideration. For efficiency, it is desirable to place the PTC heater close to the users mouth and nose to reduce conductive heat losses to the atmosphere through the ducting structure. However, the minimum hot side duct length (31) is the lower limit on how close the PTC heater can be placed to the users mouth and nose and will normally range from three to five inches. In other specific design applications, for reasons of weight, balance, or type of physical activity, the PTC heater may be located 12 to 18 inches distant from the users mouth and nose such as on the user's chest or back. Additionally, in specific application cases of a longer warm air duct after the PTC heater, the temperature averaging of the heater pulsing may occur without the use of a mixer (68).

FIG. 10 shows the top view of the personal mobile air heater architecture embodied and mounted as an add-on to a high impact helmet (78A) such as may be used on motorcycles, snowmobiles, and other recreational vehicles. A clear visor (72A) provides vision protection from debris and keeps cold air from the user's face. The PTC heater element (6) is located to the front and side of the helmet offsetting the weight from the fan (52A) and reducer section (53A) located on rear and opposite side of the helmet. Cold ambient air enters the heater system at intake screen (77A) and then filter (51A) by the action of fan (53A). The reducer section (53A) is downstream of the fan and connected to a light weight cold air duct (9A) by connector (70). The connector (70) may be quick disconnect or permanent. Cold ambient air then flows through cold air duct (9A) to the reducer fitting (12). The cold air duct (9A) is connected to the reducer fitting (12) by flange (11) and bolt (12). This connection may be quick disconnect or permanent depending on the application. The cold side air temperature is measured with sensor (54) before flowing into the PTC heater element (6). Heated air then exits the PTC heater element as a hot air pulse too hot to breath. The PTC heater element has insulation (56) arranged circumferentially around the heater to retain heat and prevent burn injuries. The hot air then flows past mixer (68) after which the hot side air temperature is measured by sensor (58). The placement of the hot side air temperature sensor (58) is sufficiently downstream that the hot and cold air pulses are mixed to a lower temperature. The breathable warm air then flows through hot side air duct (21A) and through right angle air channel (59). This warmed air flows through breathing aperture (79) and is breathable by the user inside of the helmet. Exhaled breath from the user flows down and out of the helmet through the bottom of the helmet. In extreme conditions of wind or precipitation, a flexible skirt may be added to the bottom of the helmet to reduce impingement of cold external air onto the user. The high temperature of the PTC heater element has the effect of reducing the humidity of the air flowing into the helmet. Thus, a continuous flow of warmed dry air into the helmet prevents moisture accumulation inside the helmet whether in the form of condensation or frost. The air duct (59A) may also be arranged to direct warmed air directly onto the visor to clear snow or ice accumulation.

Hot side air temperature (58), PTC hot side electrical power (18), cold side PTC electrical power (14), and cold side air temperature sensor (54) are connected in to wiring harness (60A) which is routed to the rear of the helmet. At the back of the helmet, the fan electrical power joins the wiring harness which then is routed to controller (48A). The user controls the heater operation through control knob (73A). The controller displays system attributes to the user through display (69A) arranged so that it is visible through the helmet visor, but without overly restricting visibility. Vibration mounts (75) and (76) reduce fan vibration and noise transmission into the helmet.

FIG. 11 shows the left side view of the same personal mobile air heater mounted as an add-on to a high impact helmet.

FIG. 12 shows the right side view of the same personal mobile air heater mounted as an add-on to a high impact helmet. Battery cable (91) is shown with quick disconnect (92).

The helmet with air heater system can be generally arranged on the user as shown in FIG. 13. A belt (47) holds rechargeable high-density battery (50). Suspenders (92) can be used to assist with battery weight distribution and user comfort. More than one battery may be used and arranged to distribute the weight evenly or increase operating time before battery change out is needed. The belt placement of the battery is meant to allow easy battery change out while the wearer is using the system and does not require removal of the helmet.

This system has been demonstrated to provide comfortable warm air to breath while achieving significant individual mobility free of external power or air supply. In practice, with ambient temperatures of 0 F to 32 F, system operation times of four hours for an adult male are achievable with a single battery.

FIG. 14 shows the top view of the personal mobile air heater architecture fully integrated into a custom high impact helmet (78B) such as may be used on motorcycles, snowmobiles, and other recreational vehicles. A clear visor (72B) provides vision protection from debris and keeps cold air from the user's face. The PTC heater element (6) is again located to the front and side of the helmet offsetting the weight from the fan (52B) on the rear and opposite side of the helmet. In this application, the fan (52B) is a high efficiency single stage centrifugal fan. The helmet is molded so that air intake (80) is rearward facing to avoid sucking in rain or snow while moving forward. The reducer section (53B) is molded into the helmet along with cold air duct (9B) eliminating connectors and substantially increasing the ruggedness of the system. The cold air flow is directed through PTC heater element (6) which is still placed outside of the helmet due to the amount of concentrated heat energy. Upon exiting the PTC heater element and passing through mixer (68) the warm air then flows through duct (21B) into the side of the helmet in proximity to the users mouth and nose but offset to the side. The user controls the heater operation through control knob (73B). Additional functionality such as a microphone (81) and earphone (82) connected to a radio, telephone, or intercom can be added. The wiring harness (60B) is routed over the semi-flexible surface (74) connecting all electrical components. A more sophisticated display (69B) is also used and the controller (48B) is placed inside of the helmet for ruggedness.

FIG. 15 shows the side view of the personal mobile air heater architecture embodied in a high visibility bubble helmet (72C) such as may be used in a farm or construction setting while operating heavy equipment, or other situations that place emphasis on visibility. This embodiment of the said architecture allows quick and precise movement of the users head inside of the helmet and does not restrict peripheral vision. This embodiment has a continuous semi-flexible surface (74) which goes from the user's chest, over the users shoulders, and onto the users back. This semi-flexible surface then has the system components mounted onto it which facilitates easy donning of the personal mobile system in one motion. Thus this embodiment mounts the helmet (72C) onto the top of semi-flexible surface (74), mounts the controller (48C) and battery (50) onto its front, and the fan (52C) is mounted on the rear of the semi-flexible surface. In other specific embodiments, this arrangement of components may be changed and the semi-flexible surface shaped for specific usage needs. This architecture includes all elements of the personal mobile air heater including intake screen (77C), filter (51C), fan (52C), reducer section (53C), cold air duct (9C), cold side air temperature sensor (58), PTC heater element (6), mixer (68), hot side air temperature sensor (58), warm air duct (21C), and display (69C). The wiring harness (60C) is routed over the semi-flexible surface (74) connecting all electrical components.

This embodiment is conducive to the mounting mirrors internal to the helmet heated area for rear view vision without the problems of ice, snow, or other perception accumulating on the mirrors. Similarly, this embodiment may mount recording cameras internal to the helmet heated area to avoid problems with low temperature electronics functionality or problems of ice, snow, or other precipitation accumulating on the camera lens.

FIG. 16 shows the front view of the personal mobile air heater architecture embodied in a closed mask (40) based air delivery system such as may be used in a farm, construction or other situations that place emphasis free mobility and that do not need the protection of a helmet. This embodiment can be worn under or over cold weather clothing such as jackets and caps. In this embodiment, the system components are mounted onto the upper front part of a belt (47) and suspender (92) arrangement. Thus, this embodiment mounts the controller (48D), fan (52D), intake screen (77D), filter (51D), fan (52D), reducer section (53D) onto the suspenders (92) where the user can reach them easily. In this embodiment, the warm air duct (21D) is a flexible hose which mounts to hot side air duct (21D) which in turn mounts to mask (40). The mask is held on to the users face by industry standard adjustable tension straps (57). The system control knob (73) is again placed for easy user access in the chest area. The rechargeable high-density battery (50) is worn on the belt (47). A display is not shown, but is easily placed in the vicinity of the battery or controller if desired. In other specific embodiments, this arrangement of components may be changed for specific usage needs.

FIG. 17 shows a close up front view of the closed mask (40) used with this embodiment. The warm air duct flexible hose (21D) is connected to the right angle air channel (59D) with a freely rotating ring (42). In turn (59D) is connected to the closed mask (40) with an additional freely rotating ring (34). The combination of the two rotating rings (34) and (42) greatly increases user comfort while moving around as the flexible hose offers less resistance to head movement. The mask incorporates a one-way valve (71) on the opposite side of the mask in which the users moist exhaled air volume flows out of the mask. Thus, warm and dry fresh air is flowing into the mask, while a mixture of moist exhaled air and warm dry air flows out of the mask. The warm dry air has the effect of eliminating frost and condensation inside of the mask.

FIG. 18 shows the front view of the personal mobile air heater architecture embodied in an open mask (28) based air delivery system such as may be used in sports, high intensity labor, or other high exertion situations that place emphasis free mobility and that do not need the protection of a helmet. Additionally, this embodiment is useful in situations where the user needs to audibly address people without reduction in clarity or volume. This embodiment allows inhalation and exhalation of a wide range of air volumes without any restrictions across a range of wearer respiratory needs. Warm dry air is placed in proximity to the users nose and mouth, but it is mixed with cold ambient air at the users nose and mouth. Thus, the open mask embodiment reduces overall heater efficiency to gain operation over a wide range of user exertion levels. It is light enough and flexible enough to be used for extreme weather jogging, cross-country skiing, or military operations. The opening in the mask is shown as fully open, but various designs may be incorporated which protect against wind and precipitation. Additionally, the open mask and closed mask designs can be combined into a single mask system with a removable opening on the mask for high exertions situations.

This embodiment can be worn under or over cold weather clothing such as jackets and caps. In this embodiment, the system components are mounted onto the upper front part of a belt (47) and suspender (92) arrangement. Thus, this embodiment mounts the controller (48E), fan (52E), intake screen (77E), filter (51E), fan (52E), reducer section (53E) onto the suspenders (92) where the user can reach them easily. In this embodiment, the warm air duct (21E) is a flexible hose which mounts to hot side air duct (21E) which in turn mounts to mask (28). The open mask (28) is held on to the users face by industry standard adjustable tension straps (57). The system control knob (73) is again placed for easy user access in the chest area. The rechargeable high-density battery (50) is worn on the belt (47). A display is not shown, but is easily placed in the vicinity of the battery or controller if desired. In other specific embodiments, this arrangement of components may be changed for specific usage needs.

FIG. 19 shows a close up front view of the open mask (28) used with this embodiment. The warm air duct flexible hose (21E) is connected to the right angle air channel (59E) with a freely rotating ring (42). In turn (59E) is connected to the open mask (28) with an additional freely rotating ring (34). The combination of the two rotating rings (34) and (42) greatly increases user comfort while moving around as the flexible hose offers less resistance to head movement. The mask incorporates provision for a one-way valve on the opposite side of the mask, but in this embodiment it is closed off with a cover plate (90).

FIG. 20 shows one type of a plurality of user control (48) embodiments. In this case a simple rotating knob is used with an off position to de-power the system and clockwise knob rotation turning the system power on. The rotation of the knob clockwise provides the input to the control system for the desired T_{f} set temperature. The knob settings maybe to a range of minimum and maximum settings or may be absolute temperatures. In practice, the desired T_{f} temperatures will range between 60 F to 75 F.

FIG. 21 shows a simple display (69A) for the personal mobile air heater which can be attached to a visor, breastplate, or suspender. The ambient temperature is displayed (84) along with warmed air temperature T_{f} (83), and battery charge remaining indicator (85). The display can be glued or bolted to the rest of the system in area (86). This display can be used with any of the physical embodiments of the present invention.

FIG. 22 shows a more sophisticated display (69B) with time of day (89), battery operation time remaining (88), and communication channel (87) added. This display can be used with any of the physical embodiments of the present invention. Further, various display embodiments can be used including a Bluetooth link to a smart phone or smart phone link to a remote monitor.

In conclusion, the present personal mobile air heater, in its various embodiments is a complete solution for providing warm breathable air to an individual person in a self-contained personally wearable unit. The efficiency of the present invention enables a single self-contained system to operate over a wide range of ambient temperatures while providing warmed air to the wearer for an extended period of time. Importantly, the present invention inherently eliminates the accumulation of fog, frost, or liquid water inside of the system which is a major issue in prior art. The present invention is highly adaptable to a variety of applications, manufacture, and specific designs.

1) A physical and functional architecture system for a self-contained personal mobile air heater system to provide breathable warm air comprising:
an air heater subsystem including a positive temperature coefficient semiconductor thermistor heater element used by pulsing the heating element to provide alternating hot and cold air;
a continuous airflow subsystem consisting of one or more fans causing cold ambient air to flow into the system through ducting to the air heater subsystem to be heated and delivered to the user;
a control subsystem that adapts to ambient air temperatures and pulses the positive temperature coefficient heater element to achieve the desired average air temperature for inhalation by the wearer; and
a power subsystem consisting of one or more high power density rechargeable batteries in a quick-change arrangement.

2) The system according claim 1 wherein said positive temperature coefficient semiconductor thermistor heater element is disposed in a honeycomb shape.

3) The system according to claim 1 further including a means to cause lengthwise mixing of the said alternating hot and cold air to reduce the average temperature to create breathable air temperature levels with the positive temperature coefficient semiconductor thermistor heater in close physical proximity to the users mouth and nose comprising:
an air flow mixer that creates air flow turbulence downstream of the said positive temperature coefficient heater to promote lengthwise mixing to rapidly and efficiently cool hot air to create breathable warm air; and
sufficient duct length for lengthwise turbulent flow of air causing the mixing of hot and cold air pulses to create breathable warm air.

4) The system according to claim 1 wherein the said continuous flow air supply subsystem flowing through the said pulsed positive temperature coefficient heater element provides dry air to the user and hence eliminates moisture accumulation from the users exhaled breath inside of the system.

5) The system according to claim 1 wherein the said control subsystem comprises:
a means for the user to control the system including power on-off and temperature setting;
a means to measure the temperature of both the cold ambient air temperature and the warm air temperature of the downstream flow of the air heater subsystem;
an algorithm to providing pulse power to the positive temperature coefficient semiconductor thermistor heater element which in combination with lengthwise mixing cools the hot air to create breathable warm air;
in extreme cold conditions, a control algorithm mode which can vary the hot pulse time as a control variable while treating the off time as a constant; and
in less cold conditions, a control algorithm mode which can hold the hot pulse time to a minimum constant while treating the off time as a control variable; and
a control algorithm which can use both control modes depending on ambient conditions.

6) The system according to claim 1 further comprising several types of displays for the user including, but not limited to temperatures and battery state.

7) The system according to claim 1 further comprising a means of distributing the system architecture to distribute the weight and volume of the said system to make the embodiment comfortable to wear under conditions of physical movement and exertion.

8) The system according to claim 1 further comprising the embodiment of a personal mobile air heater which adds the said invention onto an impact resistant protective helmet in contact with the user's head.

9) The system according to claim 1 further comprising the embodiment of a personal mobile air heater which integrates the invention into an impact resistant protective helmet in contact with the user's head.

10) The system according to claim 1 further comprising the embodiment of a personal mobile air heater which utilizes a wide field of view bubble helmet not in contact with the user's head.

11) The system according to claim 1 further comprising the embodiment of a personal mobile air heater which utilizes a closed mask for use in situations where the wearer has moderate exertion and does not need head protection.

12) The system according to claim 1 further comprising the embodiment of a personal mobile air heater which utilizes an open mask for use in situations where the wearer has heavy exertion and does not need head protection.

13) The system according to claim 1 further comprising an electrical architecture which utilizes the said physical architecture and architecture for the heating of cold air by use of a positive temperature coefficient semiconductor thermistor heater using pulsing to heat
cold air that is subsequently cooled to breathable temperatures all contained in a wearable mobile system which actively directs air through the heater then to the user and is actively controlled with a temperature feedback system.

14) The system according to claim 1 wherein said electrical architecture provides flexibility to the arrangement and placement of the said sub-systems in application specific embodiments including, but not limited to embodiments which are entirely self-contained inside of a helmet or embodiments in which the said subsystems are distributed over the wearers body.

15) A physical and functional architecture for a self-contained air heater system to provide warm dry air to the inside of jackets, gloves, pants, and boots using one or more positive temperature coefficient semiconductor thermistor heater elements comprising:
an air heater subsystem using a positive temperature coefficient semiconductor thermistor heater element that is pulsed to create warm air;
a continuous airflow subsystem consisting of one or more fans causing cold ambient air to flow into the system;
a control subsystem that adapts to ambient air temperatures and pulses the positive temperature coefficient heater element to achieve the desired air temperature; and
a power subsystem consisting of one or more high power density rechargeable batteries in a quick-change arrangement.

16) A physical and functional architecture for an indoor air heater system to provide warm dry air for medical needs, incubator usage, or clothes drying using one or more positive temperature coefficient semiconductor thermistor heater elements comprising:
an air heater subsystem using a positive temperature coefficient semiconductor thermistor heater element that is pulsed to create warm air;
a continuous airflow subsystem consisting of one or more fans causing cold ambient air to flow into the system;
a control subsystem that adapts to ambient air temperatures and pulses the positive temperature coefficient heater element to achieve the desired air temperature; and
a power subsystem consisting of an electrical cord plugging into facility power such as 110 volts alternating current.
